(19) United States
(12) Patent Application Publication
(10) Pub. No.: US 2019/0167611 A1
(43) Pub. Date: Jun. 6, 2019

(54) METHODS OF TREATING OR PREVENTING AMYLOID RELATED IMAGING ABNORMALITIES ASSOCIATED WITH ALZHEIMER'S DISEASE TREATMENT

(71) Applicant: BIOGEN CHESAPEAKE LLC, Cambridge, MA (US)

(72) Inventors: Sven JACOBSON, New York, NY (US); Thomas MACALLISTER, New York, NY (US)

(73) Assignee: BIOGEN CHESAPEAKE LLC, Cambridge, MA (US)

(21) Appl. No.: 16/321,240

(22) PCT Filed: Jul. 28, 2017
(86) PCT No.: PCT/US2017/044443

§ 371 Date: Jan. 28, 2019

Related U.S. Application Data
(60) Provisional application No. 62/368,375, filed on Jul. 29, 2016.

(51) Int. Cl. A61K 31/17; A61K 31/18; A61K 31/64; A61P 25/28; G01N 33/15; A61K 9/00

(52) U.S. Cl. CPC A61K 31/17; A61K 31/18; A61K 31/64; A61K 9/0053; G01N 33/15; A61K 9/0019; A61P 25/28

(57) ABSTRACT

A method of treating or preventing amyloid related imaging abnormalities (ARIA) in patients receiving one or more course of medical treatment for Alzheimer's Disease includes administrating a SUR1-TRPM4 channel inhibitor, such as glyburide, in an amount effective to prevent the formation or reduce the size of one or more ARIAs in the brain.

### CROSS-REFERENCE TO RELATED APPLICATIONS

This application is a 35 U.S.C. 371 National Phase Entry Application from PCT/US2017/044443, filed Jul. 28, 2017, which claims the benefit under 35 U.S.C. § 119(e) of the filing date of provisional patent application Ser. No. 62/368,375, filed Jul. 29, 2016, the disclosure of which is incorporated herein by reference in its entirety.

### FIELD OF THE INVENTION

This invention relates to the field of medical treatment methods, including intravenous methods of administration of drugs to a subject.

### BACKGROUND

There is a need in the field for new methods of treating or preventing amyloid related imaging abnormalities (ARIA).

### SUMMARY OF THE INVENTION

The present disclosure is drawn to methods of treating or preventing amyloid related imaging abnormalities (ARIA) in a subject undergoing at least one course of medical treatment for Alzheimer's Disease. The methods relate to administration of at least one SUR1-TRPM4 channel inhibitor in the subject in an amount effective to prevent the formation of or to reduce the size of one or more ARIAs in the brain.

In several embodiments, an effective amount of a SUR1-TRPM4 channel inhibitor, such as glyburide, is administered prior to at least on course of medical treatment for Alzheimer's. In further embodiments, the administration of a SUR1-TRPM4 channel inhibitor occurs about 6 hours or less before the initiation of the treatment. In several other embodiments, the administration of a SUR1-TRPM4 channel inhibitor occurs after the initiation of the treatment and can last for several hours. In further embodiments, the administration of a SUR1-TRPM4 channel inhibitor may occur at any time prior to, during, or following the initiation of the treatment or any combination thereof. In any embodiment, the administration may last for about 1 hours to about 96 hours or longer.

### BRIEF DESCRIPTION OF THE FIGURES

References will now be made to exemplary embodiments illustrated, and specific language will be used herein to describe the same. It will nevertheless be understood that these drawings merely depict exemplary embodiments and are not, therefore, to be considered limiting of its scope.

FIG. 1 shows the mean plasma glyburide concentration time profiles following a bolus injection and 72 hours of intravenous infusion of glyburide at two dosage levels. The data is shown on a linear scale with Plasma Glyburide Concentration in ng/mL on the vertical axis and Time in hours on the horizontal axis.

FIG. 2 shows the mean plasma glyburide concentration time profiles from the same study depicted in FIG. 1. The data is presented on a semi-logarithmic scale with Plasma Glyburide Concentration in ng/mL on the vertical logarithmic axis and Time in hours on the horizontal axis.

### DETAILED DESCRIPTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof. It is further to be understood that the embodiments disclosed in the following subsections may be combined with other embodiments from the same or other subsections without limit.

### SUR1-TRPM4 Channel Inhibitors

A SUR1-TRPM4 channel, also known as a NC_{cA-ATP} channel, is an ion channel found in the cell membranes of neurons, astrocytes, and other cells in mammals. The ion channel helps maintain the ion gradient between cells and the extracellular fluid and is associated with ion flow and concomitant fluid flow between the intercellular and extracellular space. The ion channel is comprised of subparts including a sulfonylurea receptor 1 (SUR1) and a transient receptor potential cation channel subfamily M member 4 (TRPM4).

A SUR1-TRPM4 channel inhibitor is a compound that selectively binds to the SUR1 and/or the TRPM4 subparts of the SUR1-TRPM4 channel. When the SUR1-TRPM4 channel inhibitor is bound with either part of the ion channel, the ion channel is effectively blocked or “shut-off”, resulting in fewer or no ions entering or leaving the cell through that ion channel. The fluid flow into or out of the cell is reduced or stopped due to the stabilization of the ion gradient.

SUR1-TRPM4 channel inhibitors include glyburide, 4-trans-hydroxy-glibenclamide, 3-cis-hydroxyglibenclamide, tolbutamide, chlorpropamide, tolazamide, repaglinide, nateglinide, meglitinide, midaglizole, tolazamide, gliquidone, LY397364, LY389382, glyclazide, glimepiride, 9-phenantrol, fluflenamic acid, riluzole, spermine, adenosine, quinine, quinidine, diphenylamine-2-carboxylic acid, 3′,5′-dichlorodiphenylamine-2-carboxylic acid, 5-nitro-2-(3-phenylpropyl-amino)-benzoic acid, 5-butyl-7-chloro-6-hydroxybenzo[c]-quinolizinium chloride (MPB-104), metabolites that interact with SUR1, or combinations thereof. As used herein, any reference to a SUR1-TRPM4 channel inhibitor or “channel inhibitor” is understood to be a reference to one or more of the compounds in the preceding list. Additionally, as used herein, glyburide is commonly referred to as a model SUR1-TRPM4 channel inhibitor. The reference to glyburide in specific embodiments or examples is not intended to limit the scope of the invention and is exemplary of all SUR1-TRPM4 channel inhibitors.

### Preparation of SUR1-TRPM4 Channel Inhibitor

The SUR1-TRPM4 channel inhibitors may be administered in the form of a bulk active chemical or, preferably, as a pharmaceutical composition or formulation for efficient and effective administration. Depending on the administration route and the desired dosage level, the preparation of the SUR1-TRPM4 channel inhibitor can vary for each embodiment.

For example, the SUR1-TRPM4 channel inhibitor may be prepared in lyophilized form as taught by U.S. Pat. No. 8,858,997, which is hereby incorporated by reference in its entirety. The channel inhibitor may also be prepared as a powdered composition, for example, as disclosed in U.S. Pat. No. 8,277,845, which is hereby incorporated by reference in its entirety.

In some embodiments, the channel inhibitor is prepared in a substantially liquid form suitable for intravenous injection or infusion. For example, a SUR1-TRPM4 channel inhibitor may be included in intravenous fluids containing sugars, amino acids, electrolytes, or other simple chemicals suitable for injection, which are suspended in water, Ringer's solution, U.S.P. or isotonic sodium chloride as taught by U.S. patent application Ser. No. 13/779,511, which is hereby incorporated by reference in its entirety. In such embodiments, the channel inhibitor may be suspended with or without a pharmaceutically acceptable carrier.

For oral administration, the SUR1-TRPM4 channel inhibitor may be prepared as a liquid or solid. For example, liquid forms for oral administration may include pharmaceutically acceptable emulsions, microemulsions, solutions, suspensions, syrups and elixirs as taught by U.S. patent application Ser. No. 13/779,511, which is hereby incorporated by reference in its entirety. Additionally, solid forms for oral administration may be prepared as tablets, capsules, gel capsules, or other know oral delivery systems.

In some embodiments, the channel inhibitor may be prepared as a composition containing at least one other therapeutically active or therapeutically inert compound. For example, a composition containing a SUR1-TRPM4 channel inhibitor and a substance effective to maintain blood pressure and blood glucose levels within an acceptable range may be formulated as taught by U.S. patent application Ser. No. 13/779,511, which is hereby incorporated by reference in its entirety.

### Amyloid Related Imaging Abnormalities (ARIA)

Amyloid Related Imaging Abnormalities (ARIA) are anomalies occurring in patients with Alzheimer's Disease following amyloid modifying therapy or other Alzheimer's treatment. Sperling et al., Amyloid Related Imaging Abnormalities (ARIA) in Amyloid Modifying Therapeutic Trials: Recommendations form the Alzheimer's Association Research Roundtable Workgroup, 7 ALZHEIMERS DEMENT., 4, 367-385 (July, 2011). The ARIAs distort the images produced by imaging modalities such as Magnetic Resonance Imaging (MRI), Computed Tomography (CT scan), Positron Emission Tomography (PET), or Functional Near-Infrared Spectroscopy (FNIR). Without wishing to be bound by any particular theory, ARIAs may represent vasogenic edema and/or micro-hemorrhages in the brain that are produced by a disruption of the blood brain barrier caused by or otherwise mediated by the Alzheimer's Disease treatment.

Administration of a SUR1-TRPM4 channel inhibitor is effective to prevent or treat the formation of ARIAs and to reduce the size of ARIAs. A SUR1-TRPM4 channel inhibitor closes the ion channels that are up-regulated in response to injury and mediate the pathological process, potentially through action of proteases like MMP-9. Inhibition of SUR1-TRPM4 is hypothesized to disrupt the process by down-regulating pathological mediators, such as MMP-9, and facilitating restoration of the blood brain barrier. For any administration route and for any administration duration, the SUR1-TRPM4 channel inhibitor may be administered prior to, simultaneously, or after the initiation of the Alzheimer's treatment, or a combination thereof. The SUR1-TRPM4 channel inhibitor administration may also be administered prior to or after the known presence of ARIAs in the brain. To the extent that ARIAs represent a process that can result in destruction of brain tissue, clinical side effects or deterioration may manifest. These include neurological side effects like headache, dizziness, blurred vision, loss of balance, tinnitus, fatigue, insomnia, confusion, agitation and the like, as well as clinical deterioration as measured by cognitive or other outcome measures typically used in Alzheimer's Disease. The treatment envisioned herein will have a therapeutic effect on those side effects and any clinical deterioration.

In the disclosed embodiments, the Alzheimer's Disease treatment may be any commonly used Alzheimer's treatment. For example, the Alzheimer's Disease treatment may include administration of memantine, donepezil, galantamine, rivastigmine, tacrine, combinations thereof, or similar compounds as taught by U.S. patent application Ser. No. 13/779,511, hereby incorporated by reference in its entirety. The Alzheimer's Disease treatment may include administration of amantadine, memantine, LY-450139, LY-2811376, R-fluriprofen, PBT2, tramiprosate, carmustine, morpholine, butyl amine, piperidine, cyclopentyl amine, phenyl ethylamine, 1-(2-chloroethyl)-3-hexylimidazolidin-2-one, 1-(2-chloroethyl)-3-(3-isopropylphenylimidazolidin-2-one), 1, 3-bis(2-chloroethylimidazolidin-2-one), 1-(2-chloroethyl)-3-(3-phenylimidazolidin-2-one), 1-(2-bromoethyl)-3-(2-chloroethylimidazolidin-2-one), 1-(2-chloroacetyl)-3-(2-chloroethyllimidazolidin-2-one), 1-(2-chloroethyl)-3-phenylimidazolidin-2-thione, secretase inhibitors, beta-secretase inhibitors, NMDA inhibitors, or other amyloid reduction or prevention drugs. The Alzheimer's Disease treatment may include administration of anti-amyloid antibodies, such as ACC-001, AN-1792, BIIB037, bapineuzumab, aducanumab, solanezumab, gantenerumab, anti-Aβ antibodies, monoclonal antibodies, or other antibodies that target amyloid plaques. The presence of ARIAs is most commonly discovered by the use of Magnetic Resonance Imaging (MRI), Computed Tomography (CT scan), Positron Emission Tomography (PET), or Functional Near-Infrared Spectroscopy (FNIR) imaging, and the same can be used to track the progress of the SUR1-TRPM4 treatment.

### SUR1-TRPM4 Administration

The manner and duration of administering SUR1-TRPM4 channel inhibitor may vary. Regardless of administration route, embodiments may be administered for about 1 to about 96 hours or longer. For example, the administration duration may be from about 1-5 hours, from about 5-10 hours, from about 10-15 hours, from about 15-20 hours, from about 20-25 hours, from about 25-30 hours, from about 30-35 hours, from about 35-40 hours, from about 40-45 hours, from about 45-50 hours, from about 50-55 hours, from about 55-60 hours, from about 60-65 hours, from about 65-70 hours, from about 70-75 hours, from about 75-80 hours, from about 80-85 hours, from about 85-90 hours, or from about 90-96 hours. In other embodiments, administration of the SUR1-TRPM4 channel inhibitor extends over periods of more than about 5 hours, or more than about 10 hours, or more than about 20 hours, or more than about 30 hours, or more than about 40 hours, or more than about 50 hours, or more than about 60 hours, or more than about 70 hours, or more than about 80 hours, or more than about 90 hours. In further embodiments, the SUR1-TRPM4 inhibitor administration last from about 5 hours to about 90 hours, from about 15 hours to about 80 hours, from about 25 hours to about 70 hours, or from about 35 hours to about 60 hours, or from about 45 to 50 hours.

In other embodiments, the administration may occur for an extended period of time, such as a period of about one day, or about two days, or about three days, or about four days, or about five days, or more. For patients with chronic conditions, the administration may last even longer, such as several days, or about 1 week, or about 2 weeks, or about 3 weeks, or more until symptoms subside.

In further embodiments, the SUR1-TRPM4 administration may occur about 6 hours or less prior to the initiation of the Alzheimer's treatment. As such, embodiments may begin administration about 6 hours prior to the initiation of the Alzheimer's treatment, about 5 hours prior to the initiation of the Alzheimer's treatment, about 4 hours prior to the initiation of the Alzheimer's treatment, about 3 hours prior to the initiation of the Alzheimer's treatment, about 2 hours prior to the initiation of the Alzheimer's treatment, or about 1 hour or less prior to the initiation of the Alzheimer's treatment. Such embodiments may administer the SUR1-TRPM4 channel inhibitor intermittently for a duration listed above or continuously for the duration listed above.

For any given administration duration, the administration may occur continuously or as a series of separate administrations, and also may include more than one SUR1-TRPM4 channel inhibitor and/or more than one route of administration.

In some embodiments, the SUR1-TRPM4 channel inhibitor is administered via one or more continuous infusions. A continuous infusion is an intravenous administration that may last for any of the above listed durations. In further embodiments, the administration includes at least two continuous infusions where there is about 1 to several minutes, about 1 to several hours, about 1 to several days, or about 1 to several months between the multiple continuous infusions. The at least two continuous infusions may administer the same or different SUR1-TRPM4 channel inhibitors.

In some embodiments, the SUR1-TRPM4 channel inhibitor administration is achieved by injection. An injection is an intravenous administration that may be continuous or bolus in form. A continuous injection is one that lasts for any duration stated above. A bolus injection refers to administration of the SUR1-TRPM4 channel inhibitor in a single injection that lasts for a relatively short period of time, usually a period of about 3 minutes or less. Several bolus injections may be administered in series for any of the durations disclosed above.

In further embodiments, the methods of administration include administration of the SUR1-TRPM4 channel inhibitor in a bolus injection to a subject, followed by a continuous infusion of the SUR1-TRPM4 channel inhibitor and by one or more further bolus injections of the SUR1-TRPM4 channel inhibitor. In embodiments, a second bolus injection is administered substantially immediately after the completion of the continuous infusion. For example, the second bolus administration commences less than one hour, or less than 30 minutes, or less than 10 minutes, or less than 5 minutes, or less than 3 minutes, or less than 2 minutes, or less than 1 minute, after the completion of the continuous infusion. A third bolus injection may begin after the completion of the second continuous infusion, and may begin either substantially immediately after the completion of the second continuous infusion, or may begin after an extended period of time after the completion of the second continuous infusion. Similarly, a fourth, or fifth, or other further bolus injection, and/or further continuous infusion may be administered, either substantially immediately, or after an extended period of time. It is contemplated that the entire sequence of bolus injections and continuous infusions may occur wholly prior to or after the initiation of the Alzheimer's treatment, or the sequence may be split between before, during, and after the initiation of the Alzheimer's treatment with one or more bolus injections and/or one or more continuous infusions occurring before, one or more bolus injections and/or continuous administrations occurring during, and/or one or more bolus injections and/or continuous infusions occurring after the initiation of the Alzheimer's treatment. Intravenous administration methods disclosed in U.S. Pat. No. 9,254,259 may be used, which is hereby incorporated by reference in its entirety.

In other embodiments, the SUR1-TRPM4 channel inhibitor is administered transdermally. An advantage of transdermal administration is that it may be less invasive as compared to administration by infusion or injection and may be more effective than oral pathways. For example, the SUR1-TRPM4 channel inhibitor may be administered using a transdermal patch taught in Manoj K. Mishra, Microcapsules and Transdermal Patch: A Comparative Approach for Improved Delivery of Antidiabetic Drug, 10 AAPS PHARM. SCI. TECH. 3, 928-34 (2009), which is hereby incorporated by reference in its entirely. Optionally, the drug may be administered through a transdermal gel as taught in Srinivas Mutalik & Nayanabhirama Udupa, Pharmacological Evaluation of Membrane-Moderated Transdermal Systems of Glipizide, 33 CLINICAL & EXPERIMENTAL PHARMACOLOGY & PHYSIOLOGY, 17-26 (2006), which is hereby incorporated by reference in its entirety. For example, transdermal administration may be used for subjects with chronic conditions who may benefit from continuous and/or prolonged administration of SUR1-TRPM4 channel inhibitors. The transdermal administration may occur for any duration disclosed above. In some embodiments, the transdermal administration last for about same period of weeks, months, or years that a subject will receive the amyloid modifying therapy. Administration through a transdermal patch or gel may occur prior to, simultaneously with, and/or after the initiation of the Alzheimer's treatment, or any combination thereof. In further embodiments, the transdermal administration may accompany an oral, injection, or infusion administration disclosed above or combinations thereof.

In further embodiments, the SUR1-TRPM4 channel inhibitor is administered orally. The oral administration may be via capsules, tablets, pills, powders, liquid suspension, or other commonly used oral administration forms. The oral administration may occur prior to, during, or after the initiation of the Alzheimer's treatment, or any combination thereof. In some embodiments, the oral administration occurs several times a day, daily, several times a week, weekly, or as needed in patients needing or receiving amyloid modifying therapies for around the same period of weeks, months, or years that they will receive the amyloid modifying therapy. In further embodiments, the oral administration may be combined with an injection, infusion, or transdermal administration route disclosed herein or combinations thereof.

In one embodiment, administration of SUR1-TRPM4 channel inhibitor occurs just prior to and/or during the amyloid modifying therapy and a low dosage is administered between amyloid modifying therapies. For example, a subject may receive a continuous infusion of a SUR1-TRPM4 channel inhibitor for up to 6 hours prior to the amyloid modifying therapy. The continuous infusion optionally continues throughout the therapy session and for up to a total of about 24 hours. Following the therapy, subjects may be sent home with oral or transdermal SUR1-TRPM4 channel inhibitors. The oral administration route may require patients to self-administer several times a day, daily, several times a week, weekly, as needed, or similar schedules. The transdermal administration route may require patients to wear one or more transdermal patches or apply transdermal gel for several days, weeks, or more. It is contemplated that the oral and/or transdermal administration may occur leading up to the first amyloid modifying therapy session and/or may continue following the final amyloid modifying session. The oral and/or transdermal administration will typically have a lower dose than the continuous infusion and is effective to maintain a steady state SUR1-TRPM4 channel inhibitor concentration. It is contemplated that the oral and/or transdermal dosage levels may increase leading up to and/or immediately following an amyloid modifying therapy session.

In all embodiments, the administration of the SUR1-TRPM4 channel inhibitor can be intermittent, or at gradual, continuous, constant, or controlled rates. In addition, the time of day and the number of times per hour, day, week, or month that the compounds are administered can vary depending upon desired dosages.

### Dosage Determinations

As used herein, the term “dose” and its grammatical derivatives and equivalents refers to the amount of SUR1-TRPM4 channel inhibitor administered to a subject. A dose may be described in terms of weight of a SUR1-TRPM4 channel inhibitor administered per day, in terms of the weight of SUR1-TRPM4 channel inhibitor per volume, or in equivalent types of measurements. The term “effective amount” or “effective dose” refers to the amount of a compound (e.g., a compound of the present invention) sufficient to effect beneficial or desired results. An effective amount can be administered in one or more administrations, applications or dosages and is not intended to be limited to a particular formulation or administration route.

It is understood that an effective amount of a SUR1-TRPM4 channel inhibitor as a treatment may vary depending on several factors including the specific subject receiving the administration, the administration route, the likelihood or severity of the ARIAs, the duration or amount of Alzheimer's treatment, and other procedure specific conditions. It will also be understood that dosages will vary between different SUR1-TRPM4 channel antagonists.

In one embodiment of the invention, the effective dosage level is measured in mg of SUR1-TRPM4 channel inhibitor per day and ranges from about 0.05 mg/day to about 3.0 mg/day. For example, a suitable daily dose of SUR1-TRPM4 channel inhibitor may be less than about 3.0 mg per day. For example, a suitable daily dose of glyburide may be about 2.5-3.0 mg/day, or about 2.0-2.5 mg/day, or about 1.5-2.0 mg/day, or about 1.0-1.5 mg/day, or about 0.4-1.0 mg/day, or about 0.05-0.4 mg/day. Additionally, the suitable daily dose may be about 0.05 mg/day, or about 0.25 mg/day, or about 0.5 mg/day, or about 1.0 mg/day, or about 1.5 mg/day, or about 2.0 mg/day, or about 2.5 mg/day, or about 3.0 mg/day. The effective dose for a given patient may also range from about 0.05 mg/day to about 3.0 mg/day, or from about 0.5 mg/day to about 2.5 mg/day, or from about 1.0 mg/day to about 2.0 mg/day. The SUR1-TRPM4 dosage levels are calculated in mg/day for illustrative purposes but the listed ranges are intended to include analogous dosages calculated in any weight unit per hour, day, week, month, treatment session, or similar time period.

Optionally, the dose range of the SUR1-TRPM4 channel inhibitor is an amount that yields a SUR1-TRPM4 blood plasma level of about 0.4 ng/mL to about 5 ng/mL. Suitable blood plasma concentrations include about 5 ng/mL, or about 4.5 ng/mL, or about 4 ng/mL, or about 3.5 ng/mL, or about 3 ng/mL, or about 2.5 ng/mL, or about 2 ng/mL or about 1.5 ng/mL, or about 1 ng/mL, or about 0.5 ng/mL, or similar blood plasma concentrations. In some embodiments, the suitable blood plasma concentration of the SUR1-TRPM4 inhibitor may be about 0.4-1.0 ng/mL, or about 1.0-1.5 ng/mL, or about 1.5-2.0 ng/mL, or about 2.0-2.5 ng/mL, or about 2.5-3.0 ng/mL, or about 3.0-3.5 ng/mL, or about 3.5-4.0 ng/mL, or about 4.0-4.5 ng/mL, or about 4.5-5.0 ng/mL, or combinations thereof. A suitable plasma concentration may also fall in the range of about 0.5 ng/mL to about 5.0 ng/mL, or of about 1.0 ng/mL to about 4.5 ng/mL, or of about 1.5 ng/mL to about 4.0 ng/mL, or of about 2.0 ng/mL to about 3.5 ng/mL, or of about 2.5 ng/mL to about 3.0 ng/mL. The amounts listed are intended for illustrative purposes and it is understood that any dosage levels substantially similar to those listed are covered by the present invention. The ranges are also intended to encompass analogous ranges measured in any units of weight of drug per any unit of blood plasma volume.

Optionally, the effective dose level is one that reaches a maximum SUR1-TRPM4 channel inhibitor plasma concentration level (denoted as “Cₘₐₓ”) of about 1 ng/mL to about 30 ng/mL. Suitable maximum SUR1-TRPM4 channel concentrations include about 30 ng/mL, about 28 ng/mL, about 26 ng/mL, about 24 ng/ML, about 22 ng/mL, about 20 ng/mL, about 18 ng/mL, about 16 ng/mL, about 14 ng/mL, about 12 ng/mL, about 10 ng/mL, about 8 ng/mL, about 6 ng/mL, about 4 ng/mL, about 2 ng/mL, or about 1 ng/mL, or similar concentration levels. A suitable maximum concentration level may also fall in the range of about 1-2 ng/mL, about 2-4 ng/mL, about 4-6 ng/mL, about 6-8 ng/mL, about 8-10 ng/mL, about 10-12 ng/mL, about 12-14 ng/mL, about 14-16 ng/mL, about 16-18 ng/mL, about 18-20 ng/mL, about 20-22 ng/mL, about 22-24 ng/mL, about 24-26 ng/mL, about 26-28 ng/mL, or about 28-30 ng/mL. It is understood that any dosage levels substantially similar to those listed are covered by the present invention. The ranges are intended to encompass analogous ranges measured in any units of weight of drug per any unit of volume.

Optionally, the effective dose level is one that achieves a steady-state SUR1-TRPM4 concentration of about 3.0 ng/mL to about 30.0 ng/mL. Thus, in embodiments, treatment will result in stead-state blood plasma concentrations of about 30 ng/mL, about 27 ng/mL, about 24 ng/mL, about 21 ng/mL, about 18 ng/mL, about 15 ng/mL, about 12 ng/mL, about 9 ng/mL, about 6 ng/mL, about 3 ng/mL, or anywhere between the listed concentrations. In other embodiments, the desired effective steady-state concentration may be about 3.0-5.0 ng/mL, or about 5.0-7.0 ng/mL, or about 7.0-10.0 ng/mL, or about 10.0-12.0 ng/mL, or about 12.0-14.0 ng/mL, or about 14.0-16.0 ng/mL, or about 16.0-18.0 ng/mL, or about 18.0-20.0 ng/mL, or about 20.0-22.0 ng/mL, 22.0-24.0 ng/mL, or about 24.0-26.0 ng/mL, or about 26.0-28.0 ng/mL, or about 28.0-30.0 ng/mL, or combinations thereof. In further embodiments, a steady-state concentration of about 3.0 ng/mL to about 30.0 ng/mL, or about 5.0 ng/mL to about 28.0 ng/mL, or about 7.0 ng/mL to about 26.0 ng/mL, or about 9.0 ng/mL to about 24.0 ng/mL, or about 11.0 ng/mL to about 22.0 ng/mL, or about 13.0 ng/mL to about 20.0 ng/mL, or about 15.0 ng/mL to about 18.0 ng/mL, or about 16.0 ng/mL to about 17.0 ng/mL, or combinations thereof may be desired. The desired steady-state concentration may vary depending on several factors, including the likelihood and/or severity of ARIAs, and may change over time. The ranges disclosed are exemplary and are intended to encompass analogous ranges measured in any units of weight per volume.

The specific effective dose for any particular patient will depend on a variety of factors including the severity or likelihood of the condition; activity of the specific compound employed; the age, body weight, general health, sex and diet of the patient; the preparation of the specific compound; the time and route of administration; the duration of administration; therapeutic agents used in combination or coinciding with the specific compound employed; and like factors known in the medical arts. The effective dose may also change over time as any of the ARIAs worsen or improve, the use of Magnetic Resonance Imaging (MRI), Computed Tomography (CT scan), Positron Emission Tomography (PET), or Functional Near-Infrared Spectroscopy (FNIR) can track progress and help determine dosage levels. For chronic conditions, subjects may receive an effective dose for a plurality of days, weeks, or months. The number of and frequency of administrations may vary depending upon the likelihood or severity of the ARIAs and the patient specific response to the particular SUR1-TRPM4 channel inhibitor administered.

For any compound used in the methods described herein, the effective dose may be estimated initially from cell based assays. A dosage may be formulated in animal models to achieve a desired circulating plasma concentration range. Such information can be used to more accurately determine useful doses in humans.

The foregoing description of preferred embodiments has been presented for purposes of illustration and description only. It is not intended to be exhaustive or to limit the application to the precise form disclosed, and modifications and variations are possible and/or would be apparent in light of the above teachings or may be acquired from practice of the application. The embodiments were chosen and described in order to explain the principles of the application and its practical application to enable one skilled in the art to utilize the application in various embodiments and with various modifications as are suited to the particular use contemplated. It is intended that the scope of the application be defined by the claims appended hereto and that the claims encompass all embodiments of the application, including the disclosed embodiments and their equivalents.

### EXAMPLES

Embodiments will be further described with reference to the following Examples, which are provided for illustrative purposes only and should not be used to limit the scope of or construe the invention.

### Example 1

### Proper Dosing Levels of Glyburide

To determine the proper dosage levels of SUR1-TRPM4, Glyburide was administered to 24 healthy adult subjects via a bolus injection followed by 72 hours of continuous infusion to determine the plasma concentration levels of glyburide throughout the study and the pharmacokinetic profile of the drug. Eight subjects received a bolus injection of 17.3 ng of glyburide followed by 0.4 mg/day of glyburide infusion for 72 hours. The remaining sixteen subjects received a bolus injection of 130 ng of glyburide followed by 3.0 mg/day of glyburide infusion for 72 hours. The two groups are referred to as the 0.4 mg/day group and the 3.0 mg/day group, respectively.

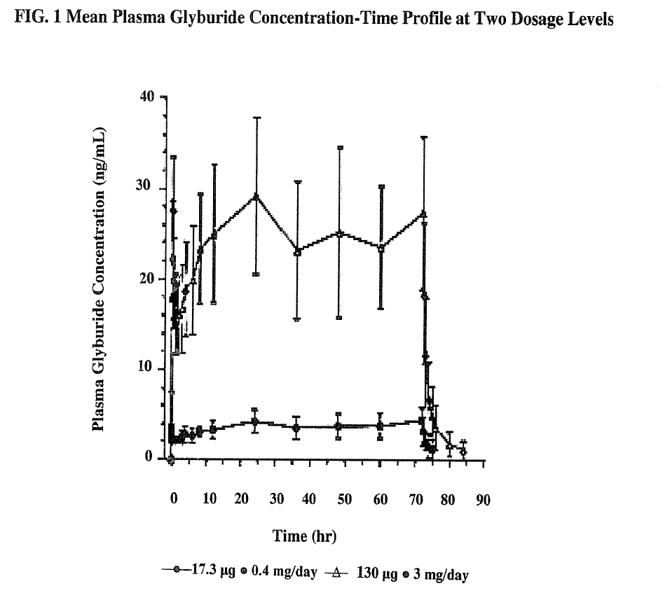

FIG. 1 shows the plasma glyburide concentration-time profiles in the subjects. The chart shows the glyburide concentrations in ng/mL on the vertical axis and the time on the horizontal axis. It is shown on a linear scale and includes the standard deviation for both the 0.4 mg/day group and the 3.0 mg/day group.

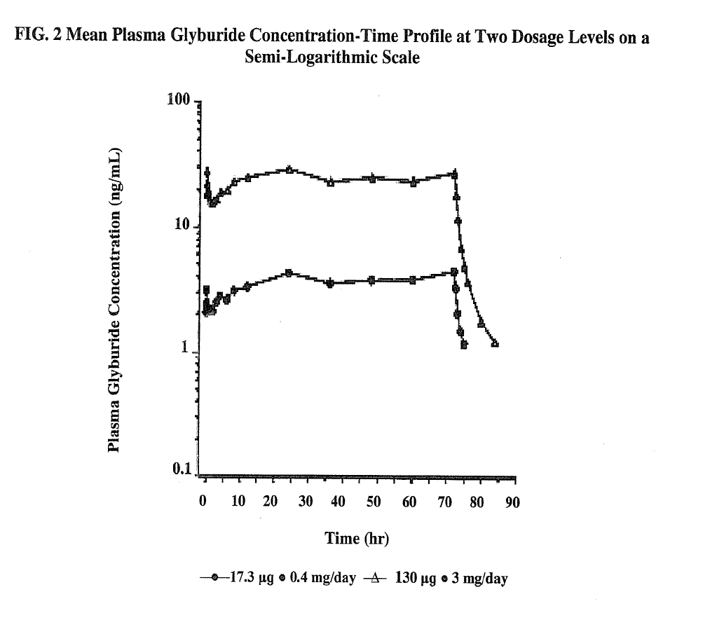

FIG. 2 shows the same plasma glyburide concentration time-profiles on a semi-logarithmic scale.

TABLE 1

| | 17.3 μg bolus + | | 130.0 μg bolus + | |
|---|---|---|---|---|
| | 0.4 mg/day | | 3.0 mg/day | |
| Pharmacokinetic | infusion for 72 hours | | infusion for 72 hours | |
| Parameter | (N = 8) | | (N = 16) | |
| Cₘₐₓ^{a} (ng/mL) | 2.983 | (0.8948) | 23.26 | (5.337) |
| AUC (hr · ng/mL) | 270 | (101) | 1779 | (536) |
| Cₛₛ (ng/mL) | 3.702 | (1.381) | 24.38 | (7.350) |
| Cₛₛ/D (ng/mL)/mg) | 3.025 | (1.127) | 2.655 | (0.8034) |
| V1, _{w} (L/kg) | 0.0925 | (0.0223) | 0.0843 | (0.0138) |
| V2, _{w} (L/kg) | 0.131 | (0.0598) | 0.0922 | (0.0201) |
| V_{ss, w} (L/kg) | 0.224 | (0.0669) | 0.176 | (0.0245) |
| V_{darea, w} (L/kg) | 0.341 | (0.178) | 0.485 | (0.274) |
| CL, _{w} (L/hr/kg) | 0.0764 | (0.0214) | 0.0837 | (0.0258) |
| CLD2, _{w} (L/hr/kg) | 0.0997 | (0.0618) | 0.0276 | (0.0164) |
| Alpha-HL (hr) | 0.346 | (0.195) | 0.540 | (0.229) |
| Beta-HL (hr) | 3.27 | (1.66) | 4.05 | (1.43) |

Source: Section 14, Table 14.7.2.
Abbreviations: N = number of subjects, D = dose, w = body weight, SD = standard deviation.
^{a}Values reported for Cmax are associated with bolus injection.

The mean steady-state glyburide concentration (Css) was 3.7 ng/mL for the 0.4 mg/day group, and 24.4 ng/mL for the 3.0 mg/day group. Steady-state was attained in accordance with the drug's terminal half-life, with no evidence of an accumulation of glyburide. Mean beta half-life (beta-HL), dose-corrected steady-state concentration (Css/D), and weight-corrected volume of the central compartment (V1, w), steady-state volume of distribution (Vss, w) and clearance (CL, w) values were similar between dose levels, consistent with dose-independent pharmacokinetics.

After the initial bolus RP-1127 IV injection, plasma M1 levels were first detected at 1 hour for the 0.4 mg/day dose group and at 10 minutes for the 3.0 mg/day dose group. Plasma M2 levels were first detectable at 2 hours for the 0.4 mg/day dose group and 20 minutes for the 3.0 mg/day dose group. Plasma M1 concentrations averaged ≤0.56 ng/mL in the 0.4 mg/day dose group and ≤4.4 ng/mL in the 3.0 mg/day dose group through 72.5 hours after the initial bolus injection. Plasma concentrations of M2 were considerably lower than those of M1, and averaged ≤0.19 ng/mL in the 0.4 mg/day dose group and ≤1.4 ng/mL in the 3.0 mg/day dose group through 72.5 hours after the initial bolus injection. Levels of the glyburide metabolites, M1 and M2, were considerably lower than those of glyburide, representing, on average, 18% and 6% of the glyburide steady-state concentration. Metabolite exposure was approximately proportional to the administered dose of RP-1127.

### Example 2

A clinical trial is performed to compare the effects of the SUR1-TRPM4 channel inhibitor glyburide administered to Alzheimer's disease patients (standard of care+glyburide) to placebo patients (standard of care).

This is a randomized, double-blind, placebo-controlled parallel group study or all active study using historical controls investigating the effect of glyburide administration to Alzheimer's disease patients who present with clinically significant ARIA.

In one arm, glyburide is administered as a bolus followed by continuous infusion.

In a second arm, glyburide is administered via a transdermal patch or gel.

In a third arm, glyburide is administered via injection.

In a fourth arm, glyburide is administered orally.

Subjects in one treatment group will initiate the infusion with 0.13 mg of CIRARA administered as a bolus over approximately 2 minutes (±1 min), followed by 0.16 mg/hr for 6 hours (±5 min), followed by 0.11 mg/hr for the remaining hours.

Total treatment duration will be a minimum of 72 hours and will continue as needed until acute brain edema has stabilized.

Glyburide is administered at 0.5, 1, 1.5, 2, 2.5, or 3.0 mg/day.

Plasma levels of glyburide are determined daily or weekly.

Each patient is monitored via blood sampling and MRI.

Magnetic resonance imaging scans monitor patients for amyloid-related imaging abnormalities and progression of edema and/or associated hemorrhage.

The following comparisons are made:

Functional outcome measures, e.g., global outcome score

Imaging measure of brain edema such as midline shift and T2 lesion volume.

Biomarker-based measures of blood brain barrier impairment such as plasma MMP-9 levels.

Brain edema or hemorrhage volume on MRI-functional recovery measures by Glasgow outcome scale-extended (GOS-E) or similar functional outcome measure.

FLAIR sequences are evaluated for the presence of ARIA. A 4-point scale is used to assess presence of ARIA.

White matter hyperintensities (WMH) are assessed using FLAIR.

The percentages of patients with ARIA are compared between treatment groups and placebo group.

The percentages of patients with ARIA are compared between treatment groups.

Changes in amyloid burden.

Rates of brain atrophy.

Changes on FDG-PET imaging.

Alzheimer's Disease Assessment Scale Cognitive subscale (ADAS-Cog),

Disability Assessment for Dementia (DAD),

Clinical Dementia Rating, mini-mental state examination, or

Clinical Global Impression of Change

Occurrence of one or more cerebral edema-related side effects:
Headache
Neck pain or stiffness
Nausea or vomiting
Dizziness
Irregular breathing
Vision loss or changes
Memory loss
Inability to walk
Difficulty speaking
Stupor
Seizures
Loss of consciousness

ktrans change

Fluid-attenuated inversion recovery (FLAIR) ratio change

Incidence of Common Terminology Criteria for Adverse Events (CTCAE)

Cerebral edema increase as measured on FLAIR volumetric imaging

Results: Compared with the control group, the patient groups administered glyburide are found to have significantly reduced or inhibited ARIA, reduced brain edema, reduced hemorrhage volume, and improved functional recovery.

### INCORPORATION BY REFERENCE

The entire disclosure of each of the patent documents and scientific articles referred to herein is incorporated by reference for all purposes.

### EQUIVALENTS

The invention disclosed herein may be embodied in other specific forms without departing from the spirit or essential characteristics thereof. The foregoing embodiments are therefore to be considered in all respects illustrative rather than limiting of the invention. The scope of the invention is thus indicated by the appended claims rather than by the foregoing descriptions, and all changes that come within the meaning and range of equivalency of the claims are intended to be embraced therein.

1. A method of treating or preventing amyloid related imaging abnormalities (ARIA) in a subject undergoing at least one course of medical treatment for Alzheimer's Disease, comprising administering a SUR1-TRPM4 channel inhibitor to the subject, wherein the SUR1-TRPM4 channel inhibitor is administered in an amount effective to prevent the formation or reduce the size of one or more ARIAs in the brain.

2. The method of claim 1, wherein the SUR1-TRPM4 channel inhibitor comprises at least one of glyburide, 4-trans-hydroxy-glibenclamide, 3-cis-hydroxyglibenclamide, tolbutamide, chlorpropamide, tolazamide, repaglinide, nateglinide, meglitinide, midaglizole, tolazamide, gliquidone, LY397364, LY389382, glyclazide, glimepiride, 9-phenantrol, fluflenamic acid, riluzole, spermine, adenosine, quinine, quinidine, diphenylamine-2-carboxylic acid, 3′,5′-dichlorodiphenylamine-2-carboxylic acid, 5-nitro-2-(3-phenylpropyl-amino)-benzoic acid, MPB-104, metabolites that interact with SUR1, or combinations thereof.

3. The method of claim 1, wherein the SUR1-TRPM4 channel inhibitor comprises glyburide.

4. The method of claim 1, wherein the one or more ARIAs are induced by an anti-beta-amyloid treatment.

5. The method of claim 1, wherein the size of one or more ARIAs in the brain is measured using at least one of Magnetic Resonance Imaging (MRI), Computed Tomography (CT scan), Positron Emission Tomography (PET), or Functional Near-Infrared Spectroscopy (FNIR).

6. The method of claim 1, wherein the SUR1-TRPM4 channel inhibitor is administered prior to at least once course of said medical treatment.

7. The method of claim 1, wherein the SUR1-TRPM4 channel inhibitor is administered about 6 hours or less prior to the initiation of said medical treatment.

8. The method of claim 1, wherein the SUR1-TRPM4 channel inhibitor is administered for about 1 hours to about 96 hours.

9. The method of claim 1, wherein at least a portion of the SUR1-TRPM4 channel inhibitor is administered during said medical treatment.

10. The method of claim 1, wherein the SUR1-TRPM4 channel inhibitor is administered via one or more continuous infusions.

11. The method of claim 1, wherein the SUR1-TRPM4 channel inhibitor is administered via a transdermal patch or gel.

12. The method of claim 1, wherein the SUR1-TRPM4 channel inhibitor is administered via injection.

13. The method of claim 1, wherein the SUR1-TRPM4 channel inhibitor is administered orally.

14. The method of claim 1, wherein the amount of the SUR1-TRPM4 channel inhibitor is about 0.05 mg/day to about 3.0 mg/day.

15. The method of claim 1, wherein the administration of the SUR1-TRPM4 channel inhibitor achieves a SUR1-TRPM4 channel inhibitor plasma level of about 0.4 ng/mL to about 5 ng/mL.

16. The method of claim 1, wherein the administration of the SUR1-TRPM4 channel inhibitor achieves a steady-state SUR1-TRPM4 channel inhibitor concentration of about 3.0 ng/mL to about 30.0 ng/mL.

17. The method of claim 1, wherein the administration of the SUR1-TRPM4 channel inhibitor achieves a Cₘₐₓ of the SUR1-TRPM4 channel inhibitor of about 1 ng/mL to about 30 ng/mL.
